SEQUENCE LISTING (12 sequences)

SEQ ID NO: 1 - DNA, 3633 bp, Homo sapiens
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgactgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 2 - protein, 1210 aa, Homo sapiens [sequence omitted]

SEQ ID NO: 3 - DNA, 51 bp, Homo sapiens
atcaaggaattaagagaagcaacatctccgaaagccaacaaggaaatcctc

SEQ ID NO: 4 - DNA, 34 bp, Homo sapiens
atcaaggaatcgaaagaccaacaaggaaatcctc

SEQ ID NO: 5 - DNA, 22 bp, Homo sapiens
aagagaagcaagagaagccctc

SEQ ID NO: 6 - DNA, 25 bp, Artificial Sequence
  Description of Artificial Sequence Synthetic primer
ccgtcgctatcaaggaatcgaaaga

SEQ ID NO: 7 - DNA, 25 bp, Artificial Sequence
  Description of Artificial Sequence Synthetic primer
ccgtcgctatcaaggaatcgaaaaa

SEQ ID NO: 8 - DNA, 25 bp, Artificial Sequence
  Description of Artificial Sequence Synthetic primer
ccgtcgctatcaaggaatcgaacga

SEQ ID NO: 9 - DNA, 28 bp, Artificial Sequence
  Description of Artificial Sequence Synthetic primer
ctatcaaggaattaagagaagcaaacct

SEQ ID NO: 10 - DNA, 28 bp, Artificial Sequence
  Description of Artificial Sequence Synthetic primer
ctatcaaggaattaagagaagcaagcct

SEQ ID NO: 11 - DNA, 27 bp, Artificial Sequence
  Description of Artificial Sequence Synthetic primer
gctatcaaggaattaagagaagcaaac

SEQ ID NO: 12 - DNA, 27 bp, Artificial Sequence
  Description of Artificial Sequence Synthetic primer
gctgtcaaggaattaagagaagcaaac

(19) United States
(12) Patent Application Publication
(10) Pub. No.: US 2014/0341884 A1
(43) Pub. Date: Nov. 20, 2014

(54) Novel Complex Mutations in the Epidermal Growth Factor Receptor Kinase Domain

(71) Applicant: Roche Molecular Systems, Inc., Pleasanton, CA (US)

(72) Inventors: Yan Li, Palo Alto, CA (US); Wei-Min Liu, Dublin, CA (US); Alison Tsan, Danville, CA (US)

(73) Assignee: Roche Molecular Systems, Inc., Pleasanton, CA (US)

(21) Appl. No.: 14/070,310

(22) Filed: Nov. 1, 2013

Related U.S. Application Data
(60) Provisional application No. 61/733,260, filed on Dec. 4, 2012.
(60) Provisional application No. 61/895,336, filed on Oct. 24, 2013.

(51) Int. Cl. C12Q 1/68; A61K 31/517; A61K 31/5377; C07K 16/28

(52) U.S. Cl. CPC C12Q 1/6886; C07K 16/2863; A61K 31/517; A61K 31/5377; C12Q 2600/156; C12Q 2600/106
USPC 424/133.1; 536/24.31; 435/6.11; 424/142.1; 514/266.4; 514/234.5; 506/9; 506/2; 506/16

(57) ABSTRACT

New mutations were found in exon 19 of the EGFR gene, the exon that is often mutated in tumors. The invention comprises methods of detecting the mutations, methods of prognosis and methods of predicting response to treatment based on the presence of absence of the mutations.

### CROSS-REFERENCE TO RELATED APPLICATIONS

This application claims priority to U.S. Provisional Application Ser. No. 61/733,260, filed on Dec. 2, 2012 and U.S. Provisional Application Ser. No. 61/895,336, filed on Oct. 24, 2013.

### SEQUENCE LISTING

The instant application contains a Sequence Listing which has been submitted electronically in ASCII format and is hereby incorporated by reference in its entirety. Said ASCII copy, created on Oct. 30, 2013, is named 31360-US1_SL.txt and is 17,618 bytes in size.

### FIELD OF THE INVENTION

The invention relates to cancer diagnostics and companion diagnostics for cancer therapies. In particular, the invention relates to the detection of mutations that are useful for diagnosis and prognosis as well as predicting the effectiveness of treatment of cancer.

### BACKGROUND OF THE INVENTION

Epidermal Growth Factor Receptor (EGFR), also known as HER1 or ErbB1, is a member of the type 1 tyrosine kinase family of growth factor receptors. These membrane-bound proteins possess an intracellular tyrosine kinase domain that interacts with various signaling pathways. Upon ligand binding, receptors in this family undergo dimerization and subsequent autophosphorylation of the tyrosine kinase domain. The autophosphorylation triggers a cascade of events in intracellular signaling pathways, including the Ras/MAPK, PI3K and AKT pathways. Through these pathways, HER family proteins regulate cell proliferation, differentiation, and survival.

A number of human malignancies are associated with aberrant expression or function of EGFR. (Mendelsohn et al., (2000), “The EGF receptor family as targets for cancer therapy,” Oncogene, 19:6550-6565.) In particular, it has been demonstrated that some cancers harbor mutations in the EGFR kinase domain (exons 18-21). In non-small cell lung cancer (NSCLC), these mutations were shown to promote anti-apoptotic pathways in malignant cells. (Pao et al. (2004). “EGF receptor gene mutations are common in lung cancers from “never smokers” and are associated with sensitivity of tumors to gefitinib and erlotinib”. P.N.A.S. 101 (36): 13306-13311; Sordella et al. (2004). “Gefitinib-sensitizing EGFR mutations in lung cancer activate anti-apoptotic pathways”. Science 305 (5687): 1163-1167.)

Therapies targeting EGFR have been developed. For example, cetuximab (ERBITUX™) and panitumumab (VECTIBIX™) are anti-EGFR antibodies. Erlotinib (TARCEVA™) and gefitinib (IRESSA™) are quinazolines useful as orally active selective inhibitors of EGFR tyrosine kinase. These drugs are most effective in patients whose cancers are driven by aberrant EGFR activity. A randomized, large-scale, double-blinded study of IRESSA™ (IRESSA Pan-Asia Study (IPASS)) compared gefitinib to the traditional chemotherapy as a first-line treatment in non-small cell lung cancer (NSCLC). (Mok et al. (2009) “Gefitinib or carboplatin paclitaxel in pulmonary adenocarcinoma.” N Eng J Med 361:947-957)). IPASS studied 1,217 patients with confirmed adeno carcinoma histology. The study revealed that progression-free survival (PFS) was significantly longer for IRESSA™ than chemotherapy in patients with EGFR mutation-positive tumors. The opposite was true for tumors where EGFR was not mutated: PFS was significantly longer for chemotherapy than IRESSA™. The study demonstrated that to improve a lung cancer patient's chances of successful treatment, EGFR mutation status must be known.

Analysis of clinical outcome revealed that patients with tumors harboring mutations in the kinase domain of EGFR (exons 18-21) have better response to erlotinib than those with tumors expressing wild-type EGFR. (U.S. Pat. Nos. 7,294,468 and 7,960,118) These mutations are predictive of response to tyrosine kinase inhibitors (TKIs) such as quinazolines erlotinib (TARCEVA™) and gefitinib (IRESSA™). Among the EGFR mutations, in-frame deletions and substitutions of nucleotides in the region of exon 19 including nucleotides 2235-2257 (corresponding to amino acids 746-753) is especially common in lung cancer patients (see U.S. Pat. No. 7,294,468 and Lynch et al. (2004) “Activating mutations in the epidermal growth factor underlying responsiveness of non-small cell lung cancer to gefitinib.” NEJM 350:2129.) These mutations are thought to result in an active kinase with altered properties, including increased susceptibility to inhibition. See Paez et al. (2004) EGFR mutations in lung cancer: correlation with clinical response to Gefitinib therapy, Science 304:1497.

Some mutations in the EGFR kinase domain are common, while others occur less frequently. However, it is essential that a clinical test for EGFR mutations target as many mutations as possible. This will assure that patients with rare mutations do not receive a “false negative” test result. If a rare mutation goes undetected, the patient with such a mutation will not receive potentially life-saving treatment. Therefore when a new mutation in the EGFR kinase domain is discovered, detecting this mutation has the potential of affecting the clinical outcome in some patients.

### SUMMARY OF THE INVENTION

In one embodiment, the invention is an isolated oligonucleotide that specifically hybridizes to a nucleic acid containing mutation 2240_{—}2264>CGAAAGA or 2252_{—}2277>GAGAAGCC in SEQ ID NO: 1. In a variation of this embodiment, the oligonucleotide comprises at least one nucleotide not matched with the natural sequence. In a further variation of this embodiment, the oligonucleotide is at least 90% identical to a sequence selected from SEQ ID NOs: 6-8 and 9-12. In a further variation of this embodiment, the oligonucleotide consists of a sequence selected from SEQ ID NOs: 6-8 and 9-12. In a further variation of this embodiment, the oligonucleotide is capable of priming selective amplification of the nucleic acid containing the mutation 2240_{—}2264>CGAAAGA in SEQ ID NO: 1 and not the non-mutant SEQ ID NO: 1. In a further variation of this embodiment the oligonucleotide is capable of priming selective amplification of the nucleic acid containing the mutation 2252_{—}2277>GAGAAGCC in SEQ ID NO: 1 and not the non-mutant SEQ ID NO: 1.

In another embodiment, the invention is a method of treating a patient having a tumor possibly harboring cells with a mutation in the epidermal growth factor receptor (EGFR) gene, comprising: testing the patient's sample for the presence of the mutated EGFR gene characterized by the mutation 2240_{—}2264>CGAAAGA or 2252_{—}2277>GAGAAGCC in SEQ ID NO: 1; and, if one of the mutations is present, administering to the patient a tyrosine kinase inhibitor compound. In a variation of this embodiment, the compound is cetuximab, panitumumab, erlotinib or gefitinib. In a further variation of this embodiment, the testing is performed using an oligonucleotide at least 90% identical to a sequence selected from SEQ ID NOs: 6-8 and 9-12. In another variation of this embodiment, the method further comprises testing the patient's sample for the presence of the mutated EGFR gene characterized by one or more of the mutations G719A, G719C, K745-A750 del K ins, E746V, E746K, L747S, E749Q, A750P, A755V, S768I, L858P, L858R, E746-R748 del, E746-S752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del AI ins, S768-V769 del SVA ins, G779S, P848L, G857V, L858R, L861Q, L883S, D896Y, 2236_{—}2248>ACCC, 2237_{—}2244>CGCCC, 2252_{—}2277>AC, 2240-2264>CGAAAGA, 2239_{—}2240 TT>CC, 2264 C>A and E746-A750 del AP ins; and in step (b), if any of the mutations is present, administering to the patient a tyrosine kinase inhibitor compound.

In another embodiment, the invention is a method of determining the likelihood of response of a cancer patient to tyrosine kinase inhibitor therapy comprising: testing the patient's sample for mutation 2240_{—}2264>CGAAAGA or 2252_{—}2277>GAGAAGCC in the EGFR gene in the patient's sample and, if the mutation is present, determining that the patient will likely respond to the tyrosine kinase inhibitor therapy. In variations of this embodiment, the tyrosine kinase inhibitor therapy comprises cetuximab, panitumumab, erlotinib or gefitinib. In further variations of this embodiment, the testing is performed using an oligonucleotide at least 90% identical to a sequence selected from SEQ ID NOs: 6-8 and 9-12. In further variations of this embodiment, the method further comprises testing the patient's sample one or more of the mutations G719A, G719C, K745-A750 del K ins, E746V, E746K, L747S, E749Q, A750P, A755V, S768I, L858P, L858R, E746-R748 del, E746-S752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del AI ins, S768-V769 del SVA ins, G779S, P848L, G857V, L858R, L861Q, L883S, D896Y, 2236_{—}2248>ACCC, 2237_{—}2244>CGCCC, 2252_{—}2277>AC, 2240-2264>CGAAAGA, 2239_{—}2240 TT>CC, 2264 C>A and E746-A750 del AP ins in the EGFR gene; and in step (b), if any of the mutations is reported as present, determining that the patient will likely respond to the tyrosine kinase inhibitor therapy.

In another embodiment, the invention is a kit for detecting mutation 2240_{—}2264>CGAAAGA or or 2252_{—}2277>GAGAAGCC in the human EGFR gene, comprising one or more oligonucleotides that specifically hybridize to the mutation 2240_{—}2264>CGAAAGA or 2252_{—}2277>GAGAAGCC in SEQ ID NO:1 but not to non-mutated SEQ ID NO:1. In variations of this embodiment, the kit of comprises an oligonucleotide at least 90% identical to a sequence selected from SEQ ID NOs: 6-8 and 9-12. In further variations of this embodiment, the kit comprises nucleic acid precursors, nucleic acid polymerase and reagents and solutions necessary to support the activity of the nucleic acid polymerase. In further variations of this embodiment, the kit comprises one or more oligonucleotides that specifically hybridize to mutations G719A, G719C, K745-A750 del K ins, E746V, E746K, L747S, E749Q, A750P, A755V, S768I, L858P, L858R, E746-R748 del, E746-S752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del AI ins, S768-V769 del SVA ins, G779S, P848L, G857V, L858R, L861Q, L883S, D896Y, 2236_{—}2248>ACCC, 2237_{—}2244>CGCCC, 2252_{—}2277>AC, 2240-2264>CGAAAGA, 2239_{—}2240 TT>CC, 2264 C>A and E746-A750 del AP ins in SEQ ID NO:1 but not to non-mutated SEQ ID NO:1.

In another embodiment, the invention is a method of treating a patient having a tumor possibly harboring cells with a mutation in the epidermal growth factor receptor (EGFR) gene, comprising: testing the patient's sample for the presence of the mutated EGFR gene characterized by the mutation 2240_{—}2264>CGAAAGA or 2252_{—}2277>GAGAAGCC in SEQ ID NO: 1; and, if the mutation is detected, administering to the patient a tyrosine kinase inhibitor compound. In variations of this embodiment, compound is cetuximab, panitumumab, erlotinib or gefitinib. In further variations of this embodiment, the testing is performed using an oligonucleotide at least 90% identical to a sequence selected from SEQ ID NOs: 6-8 and 9-12. In variations of this embodiment, the method further comprises testing the patient's sample for the presence of the mutated EGFR gene characterized by one or more of the mutations G719A, G719C, K745-A750 del K ins, E746V, E746K, L747S, E749Q, A750P, A755V, S768I, L858P, L858R, E746-R748 del, E746-S752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del AI ins, S768-V769 del SVA ins, G779S, P848L, G857V, L858R, L861Q, L883S, D896Y, 2236_{—}2248>ACCC, 2237_{—}2244>CGCCC, 2252_{—}2277>AC, 2240-2264>CGAAAGA, 2239_{—}2240 TT>CC, 2264 C>A and E746-A750 del AP ins; and in step (b), if any of the mutations is detected, administering to the patient a tyrosine kinase inhibitor compound.

### BRIEF DESCRIPTION OF THE DRAWINGS

FIG. 1 (1A-1C) shows SEQ ID NO: 1, the cDNA sequence of wild-type EGFR.

FIG. 2 shows SEQ ID NO: 2, the amino acid sequence of wild-type EGFR.

### DETAILED DESCRIPTION OF THE INVENTION

### Definitions

To facilitate the understanding of this disclosure, the following definitions of the terms used herein are provided.

The term “n_m” or “n-m del” refers to a mutation that results in a nucleic acid lacking the nucleotides between positions “n” and “m.” The term “n_m>XYZ” refers to a complex mutation where the nucleic acid is lacking nucleotides between positions “n” and “m,” but nucleotide sequence XYZ is inserted in their place. For example, the term “2239_{—}2240 TT>CC” refers to a mutation that results in a nucleic acid lacking nucleotides 2239-2240 and the nucleotide sequence CC is inserted in the place of the deleted nucleotides.

The term “allele-specific primer” or “AS primer” refers to a primer that hybridizes to more than one variant of the target sequence, but is capable of discriminating between the variants of the target sequence in that only with one of the variants, the primer is efficiently extended by the nucleic acid polymerase under suitable conditions. With other variants of the target sequence, the extension is less efficient, inefficient or undetectable.

The term “common primer” refers to the second primer in the pair of primers that includes an allele-specific primer. The common primer is not allele-specific, i.e. does not discriminate between the variants of the target sequence between which the allele-specific primer discriminates.

The terms “complementary” or “complementarity” are used in reference to antiparallel strands of polynucleotides related by the Watson-Crick base-pairing rules. The terms “perfectly complementary” or “100% complementary” refer to complementary sequences that have Watson-Crick pairing of all the bases between the antiparallel strands, i.e. there are no mismatches between any two bases in the polynucleotide duplex. However, duplexes are formed between antiparallel strands even in the absence of perfect complementarity. The terms “partially complementary” or “incompletely complementary” refer to any alignment of bases between antiparallel polynucleotide strands that is less than 100% perfect (e.g., there exists at least one mismatch or unmatched base in the polynucleotide duplex). The duplexes between partially complementary strands are generally less stable than the duplexes between perfectly complementary strands.

The term “sample” refers to any composition containing or presumed to contain nucleic acid. This includes a sample of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic acids isolated therefrom.

The terms “polynucleotide” and “oligonucleotide” are used interchangeably. “Oligonucleotide” is a term sometimes used to describe a shorter polynucleotide. An oligonucleotide may be comprised of at least 6 nucleotides, for example at least about 10-12 nucleotides, or at least about 15-30 nucleotides corresponding to a region of the designated nucleotide sequence.

The term “primary sequence” refers to the sequence of nucleotides in a polynucleotide or oligonucleotide. Nucleotide modifications such as nitrogenous base modifications, sugar modifications or other backbone modifications are not a part of the primary sequence. Labels, such as chromophores conjugated to the oligonucleotides are also not a part of the primary sequence. Thus two oligonucleotides can share the same primary sequence but differ with respect to the modifications and labels.

The term “primer” refers to an oligonucleotide which hybridizes with a sequence in the target nucleic acid and is capable of acting as a point of initiation of synthesis along a complementary strand of nucleic acid under conditions suitable for such synthesis. As used herein, the term “probe” refers to an oligonucleotide which hybridizes with a sequence in the target nucleic acid and is usually detectably labeled. The probe can have modifications, such as a 3′-terminus modification that makes the probe non-extendable by nucleic acid polymerases, and one or more chromophores. An oligonucleotide with the same sequence may serve as a primer in one assay and a probe in a different assay.

As used herein, the term “target sequence”, “target nucleic acid” or “target” refers to a portion of the nucleic acid sequence which is to be either amplified, detected or both.

The terms “hybridized” and “hybridization” refer to the base-pairing interaction of between two nucleic acids which results in formation of a duplex. It is not a requirement that two nucleic acids have 100% complementarity over their full length to achieve hybridization.

The terms “selective hybridization” and “specific hybridization” refer to the hybridization of a nucleic acid predominantly (50% or more of the hybridizing molecule) or nearly exclusively (90% or more of the hybridizing molecule) to a particular nucleic acid present in a complex mixture where other nucleic acids are also present. For example, under typical PCR conditions, primers specifically hybridize to the target nucleic acids to the exclusion of non-target nucleic acids also present in the solution. The specifically hybridized primers drive amplification of the target nucleic acid to produce an amplification product of the target nucleic acid that is at least the most predominant amplification product and is preferably the nearly exclusive (e.g., representing 90% or more of all amplification products in the sample) amplification product. Preferably, the non-specific amplification product is present in such small amounts that it is either non-detectable or is detected in such small amounts as to be easily distinguishable from the specific amplification product. Similarly, probes specifically hybridize to the target nucleic acids to the exclusion of non-target nucleic acids also present in the reaction mixture. The specifically hybridized probes allow specific detection of the target nucleic acid to generate a detectable signal that is at least the most predominant signal and is preferably the nearly exclusive (e.g., representing 90% or more of all amplification products in the sample) signal.

The present invention describes a novel mutation in the EGFR kinase domain that is useful for cancer diagnosis and prognosis, designing a therapy regimen and predicting success of the therapy.

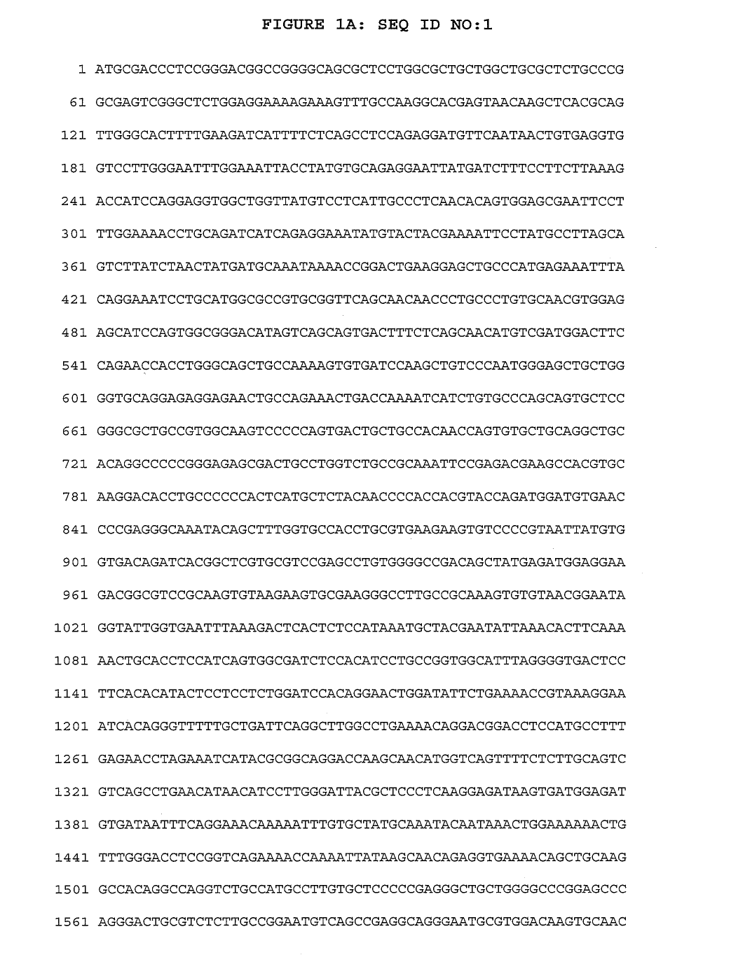

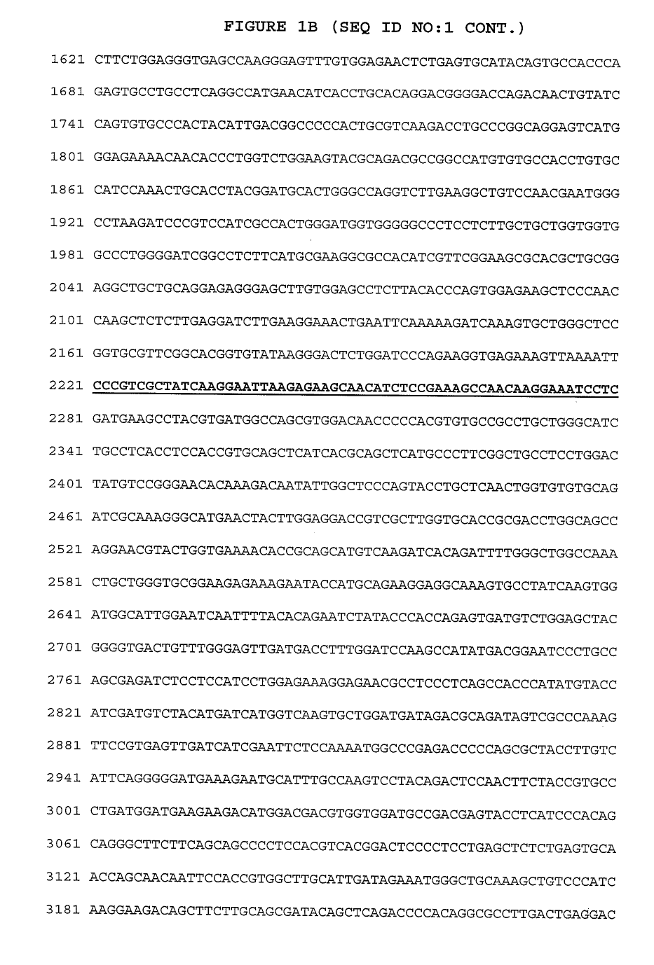

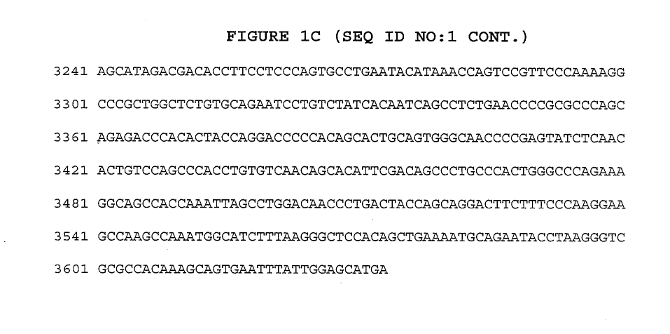

The nucleotide numbering used herein is in reference to SEQ ID NO: 1, shown on FIG. 1. Within SEQ ID NO: 1, the portion of the sequence between nucleotides 2221 and 2280, that encompasses the seven mutations described herein is highlighted and underlined.

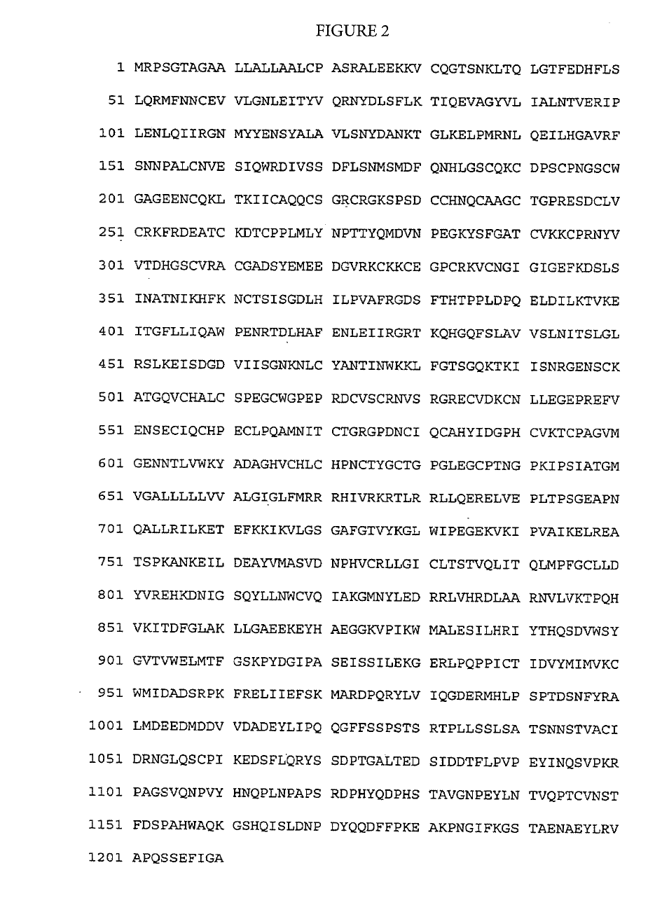

The amino acid numbering used herein is in reference to SEQ ID NO: 2, shown on FIG. 2. Within SEQ ID NO: 2, the signal sequence includes amino acids 1-24, the extracellular domain includes amino acids 24-645, the transmembrane domain includes amino acids 646-668, and the cytoplasmic domain includes amino acids 669-1210, of which the tyrosine kinase domain is amino acids 718-964, and the threonine phosphorylation site is amino acid 678.

The present invention comprises two novel mutations in the exon 19 (portion of the kinase domain) of the human EGFR gene. Mutations 2240_{—}2264>CGAAAGA and 2252_{—}2277>GAGAAGCC and the corresponding wild-type sequence are shown in Table 1.

TABLE 1
New mutations and wild-type sequence in
exon 19 of the human EGFR gene

| SEQ | | |
|---|---|---|
| ID NO: | SEQUENCE | NUCLEOTIDE SEQUENCE |
| 3 | WT 2230-2280 | ATCAAGGAATTAAGAGAAGCAACATC |
| | | TCCGAAAGCCAACAAGGAAATCCTC |
| 4 | 2240_2264> | ATCAAGGAATCGAAAGACCAACAAG |
| | CGAAAGA | GAAATCCTC |
| 5 | 2252_2277> | AAGAGAAGCAAGAGAAGCCCTC |
| | GAGAAGCC | |

In one embodiment, the present invention comprises oligonucleotides for detecting mutations 2240_{—}2264>CGAAAGA and 2252_{—}2277>GAGAAGCC in exon 19 of human EGFR gene. In variations of this embodiment, some of the oligonucleotides are allele-specific primers for use in allele-specific PCR (see U.S. Pat. No. 6,627,402). An allele-specific primer typically possesses a 3′-end matched to the target sequence (the mutant sequence) and mismatched to the alternative sequence (e.g. the wild-type sequence). Optionally, allele-specific primers may contain internal mismatches with both the wild-type and mutant target sequence. Additional mismatches in allele-specific PCR primers have been shown to increase selectivity of the primers. See U.S. patent application Ser. No. 12/582,068 filed on Oct. 20, 2009, which is incorporated herein by reference in its entirety. For successful extension of a primer, the primer needs to have at least partial complementarity to the target sequence. Generally, complementarity at the 3′-end of the primer is more critical than complementarity at the 5′-end of the primer. (Innis et al. Eds. PCR Protocols, (1990) Academic Press, Chapter 1, pp. 9-11). This means that variations of the 5′-end, i.e. additions, substitutions or removal of nucleotides at the 5′-end, do not affect performance of a primer in a PCR assay. Therefore the present invention encompasses allele-specific primers (e.g., those disclosed in Table 2) as well as their equivalents with 5′-end variations. In variations of this embodiment, the oligonucleotides are selected from Table 2 (SEQ ID NOs: 6-8 and 9-12).

TABLE 2
Allele-specific primers for
detection of mutation in EGFR

| SEQ | | MISMATCHES WITH |
|---|---|---|
| ID NO: | SEQUENCE | NATURAL SEQUENCE |
| Mutation 2240_2264>CGAAAGA | | |
| 6 | CCGTCGCTATCAAGG | none |
| | AATCGAAAGA | |
| 7 | CCGTCGCTATCAAGG | n-1 introduced A:C |
| | AATCGAAAAA | |
| 8 | CCGTCGCTATCAAGGA | n-2 introduced C:T |
| | ATCGAACGA | |
| Mutation 2252_2277>GAGAAGCC | | |
| 9 | CTATCAAGGAATTAAGA | none |
| | GAAGCAAACCT | |
| 10 | CTATCAAGGAATTAAGA | n-3 introduced G:T |
| | GAAGCAAGCCT | |
| 11 | GCTATCAAGGAATTAAGA | none |
| | GAAGCAAAC | |
| 12 | GCTGTCAAGGAATTAAGA | introduced G:T |
| | GAAGCAAAC | near 5′ |

In other variations of this embodiment, some of the oligonucleotides are detection probes specific for mutations 2240_{—}2264>CGAAAGA or 2252_{—}2277>GAGAAGCC in exon 19 of human EGFR gene. A typical mutation-specific detection probe forms a stable hybrid with the target sequence (e.g. the sequence with the mutation 2240_{—}2264>CGAAAGA or 2252_{—}2277>GAGAAGCC) and does not form a stable hybrid with the alternative sequence (e.g. the wild-type sequence at the same site) under the reaction conditions at which the detection is carried out. For successful probe hybridization, the probe needs to have at least partial complementarity to the target sequence. Generally, complementarity close to the central portion of the probe is more critical than complementarity at the ends of the probe. (Innis et al. Chapter 32, pp. 262-267). This means that variations of the ends of the probe, i.e. additions, substitutions or removal of a few nucleotides, do not affect performance of the probe in hybridization. Therefore the present invention encompasses detection probes (e.g., those disclosed in Table 2) as well as their equivalents with 5′-end and 3′-end variations. In further variations of this embodiment, the probe has a particular structure, including a protein-nucleic acid (PNA), a locked nucleic acid (LNA), a molecular beacon probe (Tyagi et al. (1996) Nat. Biotechnol. 3:303-308) or SCORPIONS® self-probing primers (Whitcombe et al. (1999) Nat. Biotechnol. 8:804-807). A probe may be labeled with a radioactive, a fluorescent or a chromophore label. For example, the mutations may be detected by real-time allele-specific polymerase chain reaction, where hybridization of a probe to the amplification product results in enzymatic digestion of the probe and detection of the digestion products (TaqMan′ probe, Holland et al. (1991) P.N.A.S. USA 88:7276-7280). Hybridization between the probe and the target may also be detected by detecting the change in fluorescence due to the nucleic acid duplex formation. (U.S. application Ser. No. 12/330,694, filed on Dec. 9, 2008) or by detecting the characteristic melting temperature of the hybrid between the probe and the target (U.S. Pat. No. 5,871,908).

Mutant EGFR gene or gene product can be detected in tumors or other body samples such as urine, sputum or serum. The same techniques discussed above for detection of mutant EGFR genes or gene products in tumor samples can be applied to other body samples. For example, cancer cells are sloughed off from tumors and appear in such body samples. State of the art nucleic acid detection methods are capable of detecting mutant cells in a background of non-tumor cells in a wide variety of sample types.

In another embodiment, the invention is a method of treating a patient having a tumor possibly harboring cells with an EGFR gene having one of the mutations 2240_{—}2264>CGAAAGA and 2252_{—}2277>GAGAAGCC in exon 19, the method comprising testing the patient's sample for the above mentioned mutation, and if the mutation is detected, administering to the patient a tyrosine kinase inhibitor (TKI) or an EGFR inhibitor. In variations of this embodiment, the tyrosine kinase inhibitors are EGFR kinase inhibitors such as for example, cetuximab, panitumumab, erlotinib or gefitinib.

In another variation of this embodiment, the method further comprises testing the patient's sample for one more of the following mutations: G719A, G719C, K745-A750 del K ins, E746V, E746K, L747S, E749Q, A750P, A755V, S768I, L858P, L858R, E746-R748 del, E746-5752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del AI ins, S768-V769 del SVA ins, G779S, P848L, G857V, L858R, L861Q, L883S, D896Y, 2236_{—}2248>ACCC, 2237_{—}2244>CGCCC, 2252_{—}2277>AC, 2240-2264>CGAAAGA, 2239_{—}2240 TT>CC, 2264 C>A and E746-A750 del AP ins; and if one or more of the mutations are present, administering to the patient a compound that inhibits signaling of the mutant EGFR protein encoded by the mutated gene. The nucleotide changes causing the mutations listed above and methods of detecting them are disclosed in U.S. Pat. Nos. 7,294,468 and 7,960,118 and U.S. application Ser. No. 13/280,976, filed on Oct. 25, 2011 (mutation E746-A750 del AP ins) U.S. application Ser. No. 13/664,333, filed on Oct. 30, 2012 (mutations 2236_{—}2248>ACCC, 2237_{—}2244>CGCCC, 2252_{—}2277>AC, 2240-2264>CGAAAGA, 22392240 TT>CC and 2264 C>A). Multiple mutations can be detected simultaneously or separately by using hybridization to multiple probes, for example in a dot-blot or nucleic acid array format, multiplex PCR, for example multiplex allele-specific PCR and multiplex PCR followed by a probe melting assay with each probe characterized by a mutation-specific melting temperature. Multiple mutations may also be detected by nucleic acid sequencing. Multiple samples can be conventiently analyzed using high-throughput sequencing for example, using a method involving emulsion PCR amplification of single molecules adhered to a solid support, subsequent sequencing by synthesis and bioinformatic analysis of the sequence data, such as the method developed by 454 Life Sciences, Inc. (Branford, Conn.) or alternative high-throughput sequencing methods and devices, e.g., ION PROTON® and PGM™, Life Technologies, Grand Island, N.Y.; HISEQ® and MISEQ®, Illumina, San Diego, Calif.).

In another embodiment, the invention is a method of determining a likelihood of response of a malignant tumor in a patient to tyrosine kinase inhibitors (TKIs) or EGFR inhibitors. The method comprises testing the patient's sample for the presence of one of the mutations 2240_{—}2264>CGAAAGA and 2252_{—}2277>GAGAAGCC in exon 19 of EGFR, and if the mutation is found, determining that the treatment is likely to be successful. In variations of this embodiment, the tyrosine kinase inhibitors are EGFR kinase inhibitors or EGFR inhibitors, for example, cetuximab, panitumumab, erlotinib or gefitinib.

In another variation of this embodiment, the method further comprises testing the patient's sample for one more of the following mutations: G719A, G719C, K745-A750 del K ins, E746V, E746K, L747S, E749Q, A750P, A755V, S768I, L858P, L858R, E746-R748 del, E746-S752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del AI ins, S768-V769 del SVA ins, G779S, P848L, G857V, L858R, L861Q, L883S, D896Y, 2236_{—}2248>ACCC, 2237_{—}2244>CGCCC, 2252_{—}2277>AC, 2240-2264>CGAAAGA, 2239_{—}2240 TT>CC, 2264 C>A and E746-A750 del AP ins; and if one or more of the mutations are present, determining that the treatment with tyrosine kinase inhibitors is likely to be successful.

In yet another embodiment, the invention is a kit containing reagents necessary for detecting one or both of the mutations 2240_{—}2264>CGAAAGA and 2252_{—}2277>GAGAAGCC in exon 19 of the human EGFR gene. The kit may comprise oligonucleotides such as probes and amplification primers specific for the mutated sequence but not the wild type sequence. In some embodiments, the kit contains at least one oligonucleotide selected from Table 2 (SEQ ID NOs: 6-8 or 9-12). In some embodiments, the kit further comprises reagents necessary for the performance of amplification and detection assay, such as the components of PCR, a real-time PCR, or transcription mediated amplification (TMA). In some embodiments, the mutation-specific oligonucleotide is detectably labeled. In such embodiments, the kit comprises reagents for labeling and detecting the label. For example, if the oligonucleotide is labeled with biotin, the kit may comprise a streptavidin reagent with an enzyme and its chromogenic substrate. In variations of this embodiment, the kit further includes reagents for detecting at least one more mutation in the EGFR gene, selected from the following: G719A, G719C, K745-A750 del K ins, E746V, E746K, L747S, E749Q, A750P, A755V, S768I, L858P, L858R, E746-R748 del, E746-S752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del AI ins, S768-V769 del SVA ins, G779S, P848L, G857V, L858R, L861Q, L883S, D896Y, 2236_{—}2248>ACCC, 2237_{—}2244>CGCCC, 2252_{—}2277>AC, 2240-2264>CGAAAGA, 2239_{—}2240 TT>CC, 2264 C>A and E746-A750 del AP ins.

In yet another embodiment, the invention is a method of treating a patient having a tumor comprising testing the patient's sample for the presence of one of the mutations 2240_{—}2264>CGAAAGA and 2252_{—}2277>GAGAAGCC in exon 19 of the EGFR gene and if the mutation is detected, administering to the patient a tyrosine kinase inhibitor (TKI). In variations of this embodiment, the tyrosine kinase inhibitor is an EGFR kinase inhibitor such as for example, cetuximab, panitumumab, erlotinib or gefitinib.

In further variations of this embodiment, the method further comprises testing the patients' sample for one more of the following mutations: G719A, G719C, K745-A750 del K ins, E746V, E746K, L747S, E749Q, A750P, A755V, S768I, L858P, L858R, E746-R748 del, E746-S752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del AI ins, S768-V769 del SVA ins, G779S, P848L, G857V, L858R, L861Q, L883S, D896Y, 2236_{—}2248>ACCC, 2237_{—}2244>CGCCC, 2252_{—}2277>AC, 2240-2264>CGAAAGA, 2239_{—}2240 TT>CC, 2264 C>A and E746-A750 del AP ins; and if one or more of the mutations are present, administering to the patient the tyrosine kinase inhibitor.

### Example 1

### Identifying the Mutations in Lung Cancer Patient Samples

Tissue samples were obtained from lung cancer (NSCLC) patients. The samples were preserved as formalin-fixed, paraffin embedded tissue (FFPET). Nucleic acids were isolated from the samples and subjected to direct sequencing on the Genome Sequencer FLX instrument according to manufacturer's instructions (454 Life Sciences, Branford, Conn.).

The 2240_{—}2264>CGAAAGA mutation was detected in the average of 26% of the total of 3,590 reads from a sample. The 2252_{—}2277>GAGAAGCC mutation was detected in the average of 21.63% of the total of 3,439 reads from a sample. Only fraction of the reads was found to contain the mutations reflecting the fact that tumors are heterogeneous and furthermore, typical samples are mixtures of tumor and non-tumor cells.

While the invention has been described in detail with reference to specific examples, it will be apparent to one skilled in the art that various modifications can be made within the scope of this invention. Thus the scope of the invention should not be limited by the examples described herein, but by the claims presented below.

What is claimed is:

1. An isolated oligonucleotide that specifically hybridizes to a nucleic acid containing mutation 2240_{—}2264>CGAAAGA or 2252_{—}2277>GAGAAGCC in SEQ ID NO: 1.

2. The oligonucleotide of claim 1, comprising at least one nucleotide not matched with the natural sequence.

3. The oligonucleotide of claim 2, at least 90% identical to a sequence selected from SEQ ID NOs: 6-8 and 9-12.

4. The oligonucleotide of claim 3 consisting of a sequence selected from SEQ ID NOs: 6-8 and 9-12.

5. The oligonucleotide of claim 3, capable of priming selective amplification of the nucleic acid containing the mutation 2240_{—}2264>CGAAAGA in SEQ ID NO: 1 and not the non-mutant SEQ ID NO: 1.

6. The oligonucleotide of claim 3, capable of priming selective amplification of the nucleic acid containing the mutation 2252_{—}2277>GAGAAGCC in SEQ ID NO: 1 and not the non-mutant SEQ ID NO: 1.

7. A method of treating a patient having a tumor possibly harboring cells with a mutation in the epidermal growth factor receptor (EGFR) gene, comprising:
(a) testing the patient's sample for the presence of the mutated EGFR gene characterized by the mutation 2240_{—}2264>CGAAAGA or 2252_{—}2277>GAGAAGCC in SEQ ID NO: 1; and,
(b) if one of the mutations is present, administering to the patient a tyrosine kinase inhibitor compound.

8. The method of claim 6, wherein said compound is cetuximab, panitumumab, erlotinib or gefitinib.

9. The method of claim 6, wherein the testing is performed using an oligonucleotide at least 90% identical to a sequence selected from SEQ ID NOs: 6-8 and 9-12.

10. The method of claim 6, further comprising in step (a), testing the patient's sample for the presence of the mutated EGFR gene characterized by one or more of the mutations G719A, G719C, K745-A750 del K ins, E746V, E746K, L747S, E749Q, A750P, A755V, S768I, L858P, L858R, E746-R748 del, E746-S752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del Al ins, S768-V769 del SVA ins, G779S, P848L, G857V, L858R, L861 Q, L883S, D896Y, 2236_{—}2248>ACCC, 2237_{—}2244>CGCCC, 2252_{—}2277>AC, 2240-2264>CGAAAGA, 2239_{—}2240 TT>CC, 2264 C>A and E746-A750 del AP ins; and in step (b), if any of the mutations is present, administering to the patient a tyrosine kinase inhibitor compound.

11. A method of determining the likelihood of response of a cancer patient to tyrosine kinase inhibitor therapy comprising:
(a) testing the patient's sample for mutation 2240_{—}2264>CGAAAGA or 2252_{—}2277>GAGAAGCC in the EGFR gene in the patient's sample and, if the mutation is present,
(b) determining that the patient will likely respond to the tyrosine kinase inhibitor therapy.

12. The method of claim 11, wherein said the tyrosine kinase inhibitor therapy comprises cetuximab, panitumumab, erlotinib or gefitinib.

13. The method of claim 11, wherein the testing is performed using an oligonucleotide at least 90% identical to a sequence selected from SEQ ID NOs: 6-8 and 9-12.

14. The method of claim 11, further comprising in step (a), further testing the patient's sample one or more of the mutations G719A, G719C, K745-A750 del K ins, E746V, E746K, L747S, E749Q, A750P, A755V, S768I, L858P, L858R, E746-R748 del, E746-S752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del Al ins, S768-V769 del SVA ins, G779S, P848L, G857V, L858R, L861 Q, L883S, D896Y, 2236_{—}2248>A000, 2237_{—}2244>CGCCC, 2252_{—}2277>AC, 2240-2264>CGAAAGA, 2239_{—}2240 TT>CC, 2264 C>A and E746-A750 del AP ins in the EGFR gene; and in step (b), if any of the mutations is reported as present, determining that the patient will likely respond to the tyrosine kinase inhibitor therapy.

15. A kit for detecting mutation 2240_{—}2264>CGAAAGA or 2252_{—}2277>GAGAAGCC in the human EGFR gene, comprising one or more oligonucleotides that specifically hybridize to the mutation 2240_{—}2264>CGAAAGA or 2252_{—}2277>GAGAAGCC in SEQ ID NO:1 but not to non-mutated SEQ ID NO:1.

16. The kit of claim 15, comprising an oligonucleotide at least 90% identical to a sequence selected from SEQ ID NOs: 6-8 and 9-12.

17. The kit of claim 15, further comprising nucleic acid precursors, nucleic acid polymerase and reagents and solutions necessary to support the activity of the nucleic acid polymerase.

18. The kit of claim 15, further comprising one or more oligonucleotides that specifically hybridize to mutations G719A, G719C, K745-A750 del K ins, E746V, E746K, L747S, E749Q, A750P, A755V, S768I, L858P, L858R, E746-R748 del, E746-S752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del Al ins, S768-V769 del SVA ins, G779S, P848L, G857V, L858R, L861Q, L883S, D896Y, 2236_{—}2248>ACCC, 2237_{—}2244>CGCCC, 2252_{—}2277>AC, 2240-2264>CGAAAGA, 2239_{—} 2240 TT>CC, 2264 C>A and E746-A750 del AP ins in SEQ ID NO:1 but not to non-mutated SEQ ID NO:1.

19. A method of treating a patient having a tumor possibly harboring cells with a mutation in the epidermal growth factor receptor (EGFR) gene, comprising:
(a) testing the patient's sample for the presence of the mutated EGFR gene characterized by the mutation 2240_{—}2264>CGAAAGA or 2252_{—}2277>GAGAAGCC in SEQ ID NO: 1; and,
(b) if the mutation is detected, administering to the patient a tyrosine kinase inhibitor compound.

20. The method of claim 19, wherein said compound is cetuximab, panitumumab, erlotinib or gefitinib.

21. The method of claim 19, wherein the testing is performed using an oligonucleotide at least 90% identical to a sequence selected from SEQ ID NOs: 6-8 and 9-12.

22. The method of claim 19, further comprising in step (a), testing the patient's sample for the presence of the mutated EGFR gene characterized by one or more of the mutations G719A, G719C, K745-A750 del K ins, E746V, E746K, L7475, E749Q, A750P, A755V, S768I, L858P, L858R, E746-R748 del, E746-S752 del V ins, L747-E749 del, L747-A750 del P ins, L747-T751 del, L747-T751 del P ins, L747-P753 del S ins, L747-S752 del, R748-P753 del, T751-I759 del T ins, S752-I759 del, P753-K757 del, M766-A767 del Al ins, S768-V769 del SVA ins, G779S, P848L, G857V, L858R, L861Q, L883S, D896Y, 2236_{—}2248>ACCC, 2237_{—}2244>CGCCC, 2252_{—}2277>AC, 2240-2264>CGAAAGA, 2239_{—}2240 TT>CC, 2264 C>A and E746-A750 del AP ins; and in step (b), if any of the mutations is detected, administering to the patient a tyrosine kinase inhibitor compound.
